(19) United States
(12) Patent Application Publication
(10) Pub. No.: US 2024/0317828 A1
(43) Pub. Date: Sep. 26, 2024

(54) T CELL ACTIVATING IMMUNOTHERAPEUTIC FOR TREATMENT OF MUCIN 1 PROTEIN EXPRESSING HUMAN CANCERS

(71) Applicant: PDS BIOTECHNOLOGY CORPORATION, Princeton, NJ (US)

(72) Inventors: Frank Bedu-Addo, Stamford, CT (US); Joseph Dervan, Doylestown, PA (US); Gregory Conn, Madrid (ES)

(21) Appl. No.: 18/381,979

(22) Filed: Oct. 19, 2023

Related U.S. Application Data
(60) Provisional application No. 63/417,640, filed on Oct. 19, 2022.

(51) Int. Cl. C07K 14/47; A61K 38/00; A61K 39/00

(52) U.S. Cl. CPC C07K 14/4727; A61K 38/00; A61K 2039/55555; A61K 2039/572; A61K 2039/585; A61K 2039/70

(57) ABSTRACT

Provided herein are multiepitope peptides including at least one mucin 1 (MUC1) peptide, the multiepitope peptides have MHC affinity for at least one of HLA serotype and are recognized by a CD4+ T cell receptor and/or by a CD8+ T cell receptor. Also provided herein are compositions comprising the multiepitope peptides and a cationic lipid, including vaccine compositions. In various aspects, the cationic lipid is R-DOTAP. The invention also provides methods of use of the multiepitope peptides and of the compositions and vaccine compositions. The methods of use include methods of treating cancer and method of inducing a MUC-specific polyfunctional cytolytic T cell response in a subject.

### CROSS-REFERENCE TO RELATED APPLICATIONS

This application claims benefit of priority under 35 U.S.C. § 119(e) of U.S. Provisional Application No. 63/417,640, filed Oct. 19, 2022. The disclosure of the prior applications is considered part of and are herein incorporated by reference in the disclosure of this application in their entirety.

### INCORPORATION OF SEQUENCE LISTING

The material in the accompanying sequence listing is hereby incorporated by reference into this application. The accompanying sequence listing xml file, name ST26.xml, was created on and is kb.

### BACKGROUND OF THE INVENTION

### Field of the Invention

The present invention relates generally to anti-cancer vaccines, and more specifically to T cell activating immunotherapeutic compositions including cationic lipids.

### Background Information

A critical component of cancer immunotherapeutic treatments is activation, amplification and targeting of cancer antigen-specific cytotoxic T-cells; these treatments play a crucial role in modulating the host immune system to recognize tumor-associated antigens and trigger cellular antitumor immune responses to eliminate targeted cancer cells. There are several approaches being developed for novel T-cell-activating cancer-targeting immunotherapeutics; 1) whole tumor cell-based immunotherapeutics, combining tumor cell components with immunostimulatory adjuvant compounds, 2) similar recombinant tumor antigen protein/peptide-based immunotherapeutics, and 3) immunotherapeutics based on DNA/RNA coding target tumor antigens. In all other previous examples developed to date the various immunostimulatory adjuvants utilized in these therapeutics, including oil in water emulsions, alum particles, and various polysaccharide and lipid components, have failed to induce strong and effective, yet safe, polyfunctional cytotoxic T cell responses in patients. There are several important hurdles that such a therapeutic cancer immunotherapeutic must overcome to induce antitumor immunity. The first hurdle is the correct choice of tumor antigen. Ideal candidates for this are antigens that are exclusively expressed in tumors, such as tumor-specific antigens (“TSA”), or antigens that are uniquely altered in the tumor cells, such as neoantigens or tumor-associated antigens (“TAA”). The second hurdle to overcome is the ability to effectively present these antigens to the dendritic cells to enable more effective presentation both via MHC I (CD8+ T cells) and MHC II (CD4 T cells). Currently utilized approaches and immune adjuvants have been ineffective in this regard. The third hurdle for an effective immunotherapeutic is to activate T cell immune responses in a broad targeted patient population expressing a wide range of HLA subtypes.

The current invention describes novel modified multi-epitope peptide formulations derived from MUC1 consisting of short and long peptides including epitope enhanced peptide agonist sequences associated with R-DOTAP immunostimulatory nanoparticles that can induce and generate high levels of MUC1 antigen-specific cytotoxic killer T-cells that recognize and kill MUC1 expressing cancer cells. The unique modified peptide antigens include long multi-epitopic peptides derived from MUC1, and specific HLA A2, A11 and A23 antigenic sequences from MUC1 whose MHC binding has been increased through amino acid substitutions and have been further modified by lipidation in order to cause formation of multi-epitopic high molecular weight micellar structures which effectively associate with R-DOTAP immunostimulatory nanoparticles for efficient delivery into dendritic cells for antigen processing yielding strong class I immune responses.

### SUMMARY OF THE INVENTION

The present invention is based on the seminal discovery of mucin-1 (herein also MUC1)-derived antigen peptides and multiepitope MUC1 peptides thereof used in vaccine compositions with cationic lipid as adjuvants to induce multifunctional anti-cancer specific T cell responses.

In one embodiment, the present invention provides a multiepitope peptide having at least 80%, 85%, 90%, or 95% sequence identity to the amino acid sequence including SEQ ID NO:1.

In one aspect, the multiepitope peptide has at least 80%, 85%, 90%, or 95% sequence identity to the amino acid sequence of any of SEQ ID NOs:9-14 and 20-37. In one aspect, the epitope peptides include at least one mucin 1 (MUC1) peptide. In one aspect, the multiepitope peptide MHC affinity for at least one of HLA-A2, HLA-A3, HLA-A11 and/or HLA-A24. In one aspect, the multiepitope peptide is recognized by a CD4+ T cell receptor and/or by a CD8+ T cell receptor. In one aspect, the multiepitope peptide has a sequence of SEQ ID NO:1. In one aspect, the multiepitope peptide is oxidized, cross-linked, pegylated, glycosylated, phosphorylated, palmitoylated, methylated, or biotinylated. In one aspect, the multiepitope peptide is palmitoylated. In one aspect, the multiepitope peptide is a cleavable anionic N-terminal sequence. In one aspect, the cleavable anionic N-terminal sequence is the amino acid sequence SSEEDE (SEQ ID NO:38) or SSEEDEE (SEQ ID NO:39).

In another embodiment, the present invention provides a composition having a multiepitope peptide and a cationic lipid, wherein the multiepitope peptide including at least one MUC-1 peptide.

In one aspect, the multiepitope peptide has at least 80%, 85%, 90%, or 95% sequence identity to the amino acid sequence including SEQ ID NO: 1. In one aspect, the at least one MUC-1 peptide has at least 80%, 85%, 90%, or 95% sequence identity to the amino acid sequence of any of SEQ ID NOs:9-14 and 20-37. In one aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP. In one aspect, the multiepitope peptide is oxidized, cross-linked, pegylated, glycosylated, phosphorylated, palmitoylated, methylated, or biotinylated. In one aspect, the multiepitope peptide has MHC affinity for at least one of HLA-A2, HLA-A3, HLA-A11 and/or HLA-A24. In one aspect, the multiepitope peptide is recognized by a CD4+ T cell receptor and/or by a CD8+ T cell receptor. In one aspect, the multiepitope peptide comprise a cleavable anionic N-terminal sequence. In one aspect, the cleavable anionic N-terminal sequence comprises the amino acid sequence SSEEDE (SEQ ID NO:38) or SSEEDEE (SEQ ID NO:39). In one aspect, the multiepitope peptide is encapsulated in liposomes comprising cationic lipids. In one aspect, the one or more multiepitope peptides and the preformed cationic lipid nanoparticles are mixed at a 1:1 ratio. In one aspect, the one or more multiepitope peptides are mixed as separate micelles with preformed cationic lipid nanoparticles. In one aspect, the composition further includes an enhancer agonist epitope and/or an analog thereof.

In an additional embodiment, the present application provides a vaccine composition having a multiepitope peptide, wherein the multiepitope has at least one mucin 1 (MUC1) peptide; and a cationic lipid.

In one aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP. In one aspect, the multiepitope peptide has a sequence comprising at least 80%, 85%, 90%, or 95% sequence identity to the amino acid sequence of SEQ ID NO:1. In one aspect, the at least one MUC-1 peptide has a sequence comprising at least 80%, 85%, 90%, or 95% sequence identity to the amino acid sequence of any of SEQ ID NOs:9-14 and 20-37. In one aspect, the multiepitope peptide has MHC affinity for at least one of HLA-A2, HLA-A3, HLA-A11 and/or HLA-A24. In one aspect, the multiepitope peptide is oxidized, cross-linked, pegylated, glycosylated, phosphorylated, palmitoylated, methylated, or biotinylated. In one aspect, the multiepitope peptide is palmitoylated. In one aspect, the multiepitope peptide has a cleavable anionic N-terminal sequence. In one aspect, the cleavable anionic N-terminal sequence has the amino acid sequence SSEEDE (SEQ ID NO:38) or SSEEDEE (SEQ ID NO:39). In one aspect, the multiepitope peptide has a sequence comprising SEQ ID NO: 1. In one aspect, the multiepitope peptide an amino acid sequence having at least 80% identity to SEQ ID NOs: 18, 19, 42 or 43. In one aspect, the multiepitope peptide is encapsulated in liposomes including cationic lipids. In one aspect, the multiepitope peptide and the preformed cationic lipid nanoparticles are mixed at a 1:1 ratio. In one aspect, the multiepitope peptide is mixed as separate micelles with preformed cationic lipid nanoparticles. In one aspect, the multiepitope peptide comprises a sequence having the amino acid sequence of SEQ ID NO: 1.

In a further embodiment, the present invention provides a method of treating cancer in a subject including administering to the subject a vaccine composition having a multiepitope peptide, wherein the multiepitope peptide has at least one mucin 1 (MUC1) peptides; and a cationic lipid.

In one aspect, the multiepitope peptide has a sequence comprising at least 80%, 85%, 90% or 95% identity to the amino acid sequence of SEQ ID NO:1. In one aspect, the at least one MUC1 peptide has a sequence comprising at least 80%, 85%, 90% or 95% identity to the amino acid sequence comprising any of SEQ ID NOs:9-14 and 20-37. In one aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP. In one aspect, the multiepitope peptide is encapsulated in cationic lipid nanoparticles. In one aspect, the multiepitope peptide and the preformed cationic lipid nanoparticles are mixed at a 1:1 ratio. In one aspect, the multiepitope peptide is mixed as separate micelles with preformed cationic lipid nanoparticles. In one aspect, the one or more MUC1 peptides induce the presentation of non-HLA restricted peptides to CD4⁺ and CD8⁺ T cells by antigen presenting cells. In one aspect, treating cancer includes preventing progression of cancer in the subject. In one aspect, the cancer has MUC1 expressing cancer cells. In one aspect, the cancer is prostate cancer, breast cancer or acute myeloid leukemia (AML). In one aspect, the method further comprising administering to the subject an anti-cancer treatment. In one aspect, the anti-cancer treatment includes an immune checkpoint inhibitor therapy. In one aspect, treating cancer includes inducing a MUC-specific polyfunctional cytolytic T cell response in the subject.

In one embodiment, the present invention provides a method of inducing a MUC-specific polyfunctional cytolytic T cell response in the subject by administering to the subject a composition including a multiepitope peptide, wherein the multiepitope peptide has one or more mucin 1 (MUC1) peptides; and a cationic lipid.

In one aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP. In one aspect, the multiepitope peptide is encapsulated in cationic lipid nanoparticles. In one aspect, the multiepitope peptide and the preformed cationic lipid nanoparticles are mixed at a 1:1 ratio. In one aspect, the multiepitope peptide is mixed as separate micelles with preformed cationic lipid nanoparticles. In one aspect, the multiepitope peptide induces the presentation of non-HLA restricted peptides to CD4⁺ and CD8⁺ T cells by antigen presenting cells.

### BRIEF DESCRIPTION OF THE DRAWINGS

FIG. 1 is a graph illustrating HLA-A2 specific CD8 T cell induction in response to peptide antigens derived from MUC1 proteins incorporated into R-DOTAP nanoparticles.

FIG. 2 is a graph illustrating HLA-A2 specific CD8 T cell induction in response to peptide antigens derived from MUC1 proteins incorporated into R-DOTAP nanoparticles.

FIG. 3 is a graph illustrating HLA-A2 specific CD8 T cell responses to formulations including peptide antigen C1A (SEQ ID NO:9) and C2A (SEQ ID NO:10) incorporated into the lipid bilayer of R-DOTAP nanoparticles.

FIG. 4 is a graph illustrating HLA-A2 specific CD8 T cell responses to formulations including peptide antigen SSEEDE-C1A (SEQ ID NO:18) and SSEEDE-C2A (SEQ ID NO:19) formulated with R-DOTAP nanoparticles and a micellar mixture with six lipidated peptide agonist antigens, pC3A: (SEQ ID NO:15), pV1A: (SEQ ID NO:SEQ ID NO:2), pV2A: (SEQ ID NO:SEQ ID NO:3), pC5A: (SEQ ID NO:SEQ ID NO:4), pC6A: (SEQ ID NO:SEQ ID NO:5), pP93L: (SEQ ID 016).

FIG. 5 is a graph illustrating HLA-A2 specific CD8 T cell responses to formulations including peptide antigen SSEEDE-C1A (SEQ ID NO:9) and SSEEDE-C2A (SEQ ID NO:10) formulated with R-DOTAP nanoparticles and a micellar mixture with six lipidated peptide agonist antigens, pC3A: (SEQ ID NO: 15), pV1A: (SEQ ID NO:2), pV2A: (SEQ ID NO:3), pC5A: (SEQ ID NO:4), pC6A: (SEQ ID NO:5), pP93L: (SEQ ID NO:16).

FIG. 6 is a graph illustrating HLA-A2 specific CD8 T cell responses to MUC1/RDOTAP vaccine formulations containing long MUC1 peptide antigen YL-40, containing the C1A (SEQ ID NO:2) and C2A (SEQ ID NO:3) antigens.

FIG. 7 is a graph illustrating HLA-A2 specific CD8 T cell responses to formulations including peptide antigen C1A (SEQ ID NO:9) and C2A (SEQ ID NO:10) incorporated into the lipid bilayer of R-DOTAP nanoparticles.

FIG. 8 is a graph illustrating HLA-A2 specific CD8 T cell responses to formulations including peptide antigen C1A (SEQ ID NO:9) and C2A (SEQ ID NO:10) mixed or encapsulated into the lipid bilayer of R-DOTAP nanoparticles.

### DETAILED DESCRIPTION

The present invention is based on the seminal discovery of mucin-1-derived antigen peptides and multiepitope peptides thereof used in vaccine compositions with cationic lipid as adjuvants to induce multifunctional anti-cancer specific T cells responses.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

Mucin 1 (also known as CD227, episialin, PEM, H23Ag, EMA, CA15-3, and MCA) is a transmembrane glycoprotein aberrantly expressed on many epithelial cancer cells and varies in its cellular distribution, function, and glycosylation. The protein is a heterodimer with a large extracellular domain covalently bound to a small intracellular domain. The extracellular domain consists of a large number of tandem repeat regions (VNTR) and non-tandem regions. The C-terminus domain of MUC1 consists of interaction sites for several signaling molecules and has been shown to have oncogenic potential.

Human clinical trials testing Mucin 1 (MUC1) as a tumor associated antigen therapeutic target have used several approaches to generate cytotoxic T cells capable of killing MUC1 expressing cancer cells. These clinical trials included use of MUC1 polypeptides, DNA sequences or viral vectors consisting of MUC1 N-terminus immunogenic VNTR region and non-VNTR region derived T cell epitopes to generate cytotoxic T-cells. None of these approaches were able to meet clinical benefit criteria due to their inability to generate sufficient T cells capable of killing MUC1 expressing cancer cells. Current approaches target sequences from the C-Terminus of MUC1 and peptide modifications to generate epitope enhanced polypeptides that can improve vaccine immunogenicity.

Cross presentation refers to an immunological pathway in which soluble proteins or peptides enter the cell from the outside and enter into the MHC class I processing pathway. This can occur in two ways, via the cytosolic pathway or the endosomal pathway. In both pathways, the peptides/proteins are initially taken up in endosomes/phagosomes. In the cytosolic pathway, a portion of partially degraded endosomal protein(s) ultimately enters the cytoplasm, where they are processed through proteasomes and the resulting peptides transported by transporter associated with Ag-processing (TAP) into either the endoplasmic reticulum or other endosomes for binding to MHC class I. Alternatively, proteins can be endosomally degraded, and peptides can bind to MHC class I present in the endosomes. This latter pathway is proteasome-independent and inefficient as it relies on the chance production of the correct peptide by endosomal proteases. Entry of proteins into early endosomes, which contain limited proteolytic activity, favor cross-presentation, while late endosomes, which contain higher levels of proteolytic activity, may inhibit cross-presentation.

Certain specific enantiomeric cationic lipids are unique in their ability to rapidly bind to antigen-presenting dendritic cells in a receptor-independent fashion and be taken up into early endosomes along with associated peptide or protein antigens. We demonstrated that these specific cationic lipids are capable of facilitating orders of magnitude more protein and peptide antigens entry into the MHC class I and MHC class II pathways than other current approaches such as the use of alum or oil in water adjuvants to induce dendritic cell maturation. Furthermore, vaccination with these peptide-antigen associated cationic lipid nanoparticles induces superior T cell immune responses in vivo compared to peptide alone or peptides formulated with traditional adjuvants.

In one embodiment, the present invention provides a multiepitope peptide having at least 80%, 85%, 90%, or 95% sequence identity to the amino acid sequence including SEQ ID NO:1.

For example, the multiepitope peptide has at least 80% sequence identity to the amino acid sequence including SEQ ID NO:1. The multiepitope peptide has at least 85% sequence identity to the amino acid sequence including SEQ ID NO:1. The multiepitope peptide has at least 90% sequence identity to the amino acid sequence including SEQ ID NO:1. The multiepitope peptide has at least 91% sequence identity to the amino acid sequence including SEQ ID NO:1. The multiepitope peptide has at least 92% sequence identity to the amino acid sequence including SEQ ID NO:1. The multiepitope peptide has at least 93% sequence identity to the amino acid sequence including SEQ ID NO: 1. The multiepitope peptide has at least 94% sequence identity to the amino acid sequence including SEQ ID NO: 1. The multiepitope peptide has at least 95% sequence identity to the amino acid sequence including SEQ ID NO: 1. The multiepitope peptide has at least 96% sequence identity to the amino acid sequence including SEQ ID NO:1. The multiepitope peptide has at least 97% sequence identity to the amino acid sequence including SEQ ID NO:1. The multiepitope peptide has at least 98% sequence identity to the amino acid sequence including SEQ ID NO:1. The multiepitope peptide has at least 99% sequence identity to the amino acid sequence including SEQ ID NO:1. The multiepitope peptide has at least 99.5% sequence identity to the amino acid sequence including SEQ ID NO:1. The multiepitope peptide has at least 99.9% sequence identity to the amino acid sequence including SEQ ID NO:1.

The terms “peptide”, “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence (e.g., SEQ ID NOs: 1-37) aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 85%, at least 90%, at least 95%, at least 96%, at least 97%, at least 98%, at least 99%, at least 99.5%, or at least 99.9%. In general, an exact match indicates 100% identity over the length of the reference sequence (e.g., SEQ ID NOs: 1-43).

Polypeptides and polynucleotides that are about 85, 86, 87, 88, 89, 90, 91, 92, 93, 94, 95, 96, 97, 98, 99 99.5% or more identical to polypeptides and polynucleotides described herein are embodied within the disclosure. For example, a polypeptide can have 80%, 85%, 90%, 91%, 92%, 93%, 94%, 95%, 96%, 97%, 98%, 99% or more identity to SEQ ID NOs: 1-43.

Variants of the disclosed sequences also include peptides, or full-length protein, that contain substitutions, deletions, or insertions into the protein backbone, that would still leave at least about 70% homology to the original protein over the corresponding portion. A yet greater degree of departure from homology is allowed if like-amino acids, i.e., conservative amino acid substitutions, do not count as a change in the sequence. Examples of conservative substitutions involve amino acids that have the same or similar properties. Illustrative amino acid conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

As used herein, the terms “polyepitope peptide”, “multi-epitope peptide” and the like refer to peptide or polypeptide that includes at least two epitopes as described herein. For example, the polyepitope peptide includes 2, 3, 4, 5, 6, 7, 8, 9, 10 or more of the epitopes of the invention.

The term “epitope” refers to an antigenic determinant in a molecule such as an antigen, i.e., to a part in or fragment of the molecule that is recognized by the immune system. An epitope of a protein such as a tumor antigen preferably comprises a continuous or discontinuous portion of said protein. The terms “epitope”, “antigen peptide”, “antigen epitope”, “immunogenic peptide”, “antigenic fragment” and “MHC binding peptide” can be used interchangeably herein and preferably relate to a representation of an antigen which is capable of eliciting an immune response against the antigen or a cell expressing or comprising and preferably presenting the antigen. An “antigen” according to the invention covers any substance that will elicit an immune response. In particular, an “antigen” relates to any substance, preferably a peptide or protein, that reacts specifically with antibodies or T-lymphocytes (T cells). According to the present invention, the term “antigen” comprises any molecule which comprises at least one epitope. Preferably, an antigen in the context of the present invention is a molecule which, optionally after processing, induces an immune reaction. According to the present invention, any suitable antigen may be used, which is a candidate for an immune reaction, wherein the immune reaction is preferably a cellular immune reaction. In the context of the embodiments of the present invention, the antigen is preferably presented by a cell, preferably by an antigen presenting cell which includes a diseased cell, in particular a cancer cell, in the context of MHC molecules, which results in an immune reaction against the antigen. An antigen is preferably a product which corresponds to or is derived from a naturally occurring antigen. Such naturally occurring antigens include tumor antigens.

The epitopes referred to in the present application include any epitope that can be derived from a MUC1 peptide. That includes for examples, epitopes having the amino acid sequences of SEQ ID NO: 1.

In another aspect, the multiepitope peptides include mucin 1 (MUC1) peptides.

In one aspect, the MUC1 peptides have a sequence having at least 80%, 85%, 90% or 95% identity with the sequence of any of SEQ ID NOs: 9-14 and 20-37.

For example, the MUC1 peptides have a sequence having at least 80% identity with the sequence of any of SEQ ID NOs: 9-14 and 20-37.

The MUC1 peptides have a sequence having at least 85% sequence identity with the sequence of any of SEQ ID NOs: 9-14 and 20-37. The MUC1 peptides have a sequence having at least 90% sequence identity with the sequence of any of SEQ ID NOs: 9-14 and 20-37. The MUC1 peptides have a sequence having at least 91% sequence identity with the sequence of any of SEQ ID NOs: 9-14 and 20-37. The MUC1 peptides have a sequence having at least 92% sequence identity with the sequence of any of SEQ ID NOs: 9-14 and 20-37. The MUC1 peptides have a sequence having at least 93% sequence identity with the sequence of any of SEQ ID NOs: 9-14 and 20-37. The MUC1 peptides have a sequence having at least 94% sequence identity with the sequence of any of SEQ ID NOs: 9-14 and 20-37. The MUC1 peptides have a sequence having at least 95% sequence identity with the sequence of any of SEQ ID NOs: 9-14 and 20-37. The MUC1 peptides have a sequence having at least 96% sequence identity with the sequence of any of SEQ ID NOs: 9-14 and 20-37. The MUC1 peptides have a sequence having at least 97% sequence identity with the sequence of any of SEQ ID NOs: 9-14 and 20-37. The MUC1 peptides have a sequence having at least 98% sequence identity with the sequence of any of SEQ ID NOs: 9-14 and 20-37. The MUC1 peptides have a sequence having at least 99% sequence identity with the sequence of any of SEQ ID NOs: 9-14 and 20-37. The MUC1 peptides have a sequence having at least 99.5% sequence identity with the sequence of any of SEQ ID NOs: 9-14 and 20-37. The MUC1 peptides have a sequence having at least 99.9% sequence identity with the sequence of any of SEQ ID NOs: 9-14 and 20-37.

As noted above, peptide epitopes, such as MUC1 epitopes can have affinity with different MHC molecules expressed by antigen presenting cells (APCs), which in turn indicates which immune cell receptors can be presented those peptides epitopes, which immune cell receptor recognize it, and therefore which type of immune response can be induced by said peptide epitopes. As detailed in the Examples section the MUC1 epitopes describes herein have affinity for several HLA molecules and are therefore capable of inducing several immune response types.

In some aspects, the MUC1 epitopes have MHC affinity for HLA-A2, A3, A11 and A24. In various aspects, the MUC1 epitopes are recognized by a CD4+ T cell receptor and/or by a CD8+ T cell receptor.

As used herein, a peptide epitope that is “recognized by” an immune cell receptor can interchangeably be refers to as a “immune cell receptor epitope”. That is, a MUC1 epitope that is recognized by a CD4+ T cell receptor can be referred to as a CD4+ T cell receptor epitope or to a MUC1 epitope, depending on the emphasis made on the peptide from which the epitope is derived from (e.g., MUC1) or on the immune cell receptor that it can be recognized by (or interact with based on affinity), (e.g., a CD4+ T cell receptor and/or a CD8+ T cell receptor).

In one aspect, the multiepitope peptide includes an amino acid sequence including at least 80%, 85%, 90% or 95% sequence identity to SEQ ID NO:1.

In another aspect, the one or more epitope peptides are oxidized, cross-linked, pegylated, glycosylated, phosphorylated, palmitoylated, methylated, or biotinylated. In various aspects, the one or more epitope peptides are palmitoylated.

In one aspect, the one or more epitope peptides include a cleavable anionic N-terminal extension. In some aspects, the cleavable anionic N-terminal extension includes the amino acid sequence SSEEDE (SEQ ID NO:38).

In another aspect, the cleavable anionic N-terminal sequence is the amino acid sequence SSEEDEE (SEQ ID NO:39). SEQ ID NO:38 and SEQ ID NO:39 are variations of the anion tag, which can be used alternatively. Notably any sequences provided herein that include an anion tag is meant to be disclosed as including the variation of the anion tag. For example, the disclosure of SEQ ID NO:18, SSEEDE-YLAIVYLIAL comprising the anion tag of SEQ ID NO:38 is meant to include the disclosure of a modified peptide including the anion tag of SEQ ID NO: 39. Non-limiting examples of such alternatively modified peptides include SEQ ID NO:42 and SEQ ID NO:43.

In another embodiment, the invention provides a composition comprising one or more of the multiepitope peptides described herein and a cationic lipid.

As used herein the term composition is meant to include pharmaceutical compositions, which may also contain other therapeutic agents, and may be formulated, for example, by employing conventional pharmaceutically acceptable vehicles or diluents, as well as pharmaceutical additives of a type appropriate to the mode of desired administration (for example, excipients, preservatives, etc.) according to techniques known in the art of pharmaceutical formulation. In certain embodiments, the compositions disclosed herein are formulated with additional agents that promote entry into the desired cell or tissue. Such additional agents include micelles, liposomes, and dendrimers.

The term “pharmaceutically acceptable” refers to the fact that the carrier, diluent or excipient must be compatible with the other ingredients of the formulation and not deleterious to the recipient thereof. For example, the carrier, diluent, or excipient or composition thereof may be administered to a subject along with a conjugate of the invention without causing any undesirable biological effects or interacting in an undesirable manner with any of the other components of the pharmaceutical composition in which it is contained.

Pharmaceutical compositions including the peptides or compositions described herein may be administered by any suitable means, for example, parenterally, such as by subcutaneous, intravenous, intramuscular, intrathecal, or intracisternal injection or infusion techniques (e.g., as sterile injectable aqueous or non-aqueous solutions or suspensions) in dosage formulations containing non-toxic, pharmaceutically acceptable vehicles or diluents. Depending on the condition being treated, these pharmaceutical compositions may be formulated and administered systemically or locally. Techniques for formulation and administration are generally known in the art. Suitable routes may, for example, parenteral delivery, including intramuscular, subcutaneous, intramedullary, intrathecal, intraventricular, intravenous, or intraperitoneal. For injection, the pharmaceutical compositions of the invention may be formulated in aqueous solutions, preferably in physiologically compatible buffers such as water, Hanks' solution, Ringer's solution, or physiologically buffered saline.

Adjuvants are often used to modify or augment the effects of a vaccine by stimulating the immune system to respond to the vaccine more vigorously, and thus providing increased immunity to a particular disease. Adjuvants accomplish this task by mimicking specific sets of evolutionarily conserved molecules, so called pathogen-associated molecular patterns, which include liposomes, lipopolysaccharide, molecular cages for antigens, components of bacterial cell walls, and endocytosed nucleic acids such as RNA, double-stranded RNA, single-stranded DNA, and unmethylated CpG dinucleotide-containing DNA. Because immune systems have evolved to recognize these specific antigenic moieties, the presence of an adjuvant in conjunction with the vaccine can greatly increase the innate immune response to the antigen by augmenting the activities of dendritic cells, lymphocytes, and macrophages by mimicking a natural infection.

The composition described herein can be formulated with a lipid nanoparticle as an adjuvant to enhance the presentation of the antigens to antigen presenting cells, and therefore to increase the immune response induce by the antigens.

In some aspects described herein, the adjuvant is a cationic lipid. As used herein, the term “cationic lipid” refers to any of a number of lipid species which carry a net positive charge at physiological pH or have a protonatable group and are positively charged at pH lower than the pKa.

Suitable cationic lipids according to the present disclosure include, but are not limited to: 3-β [4NIN, 8-diguanidino spermidine)-carbamoyl]cholesterol (BGSC); 3-β [N,N-diguanidinoethyl-aminoethane)-carbamoyl]cholesterol (BGTC); N,N.IN2N3Tetra-methyltetrapalmitylspermine (cellfectin); N-t-butyl-N′-tetradecyl-3-tetradecyl-aminopropion-amidine (CLONfectin); dimethyldioctadecyl ammonium bromide (DDAB); 1,2-dimyristyloxypropyl-3-dimethyl-hydroxy ethyl ammonium bromide (DMRIE); 2,3-dioleoyloxy-N-[2(sperminecarboxamido)ethyl]-N,N-dimethyl-1-p- -ropanaminium trifluorocetate) (DOSPA); 1,3-dioleoyloxy-2-(6-carboxyspermyl)-propyl amide (DOSPER); 4-(2,3-bis-palmitoyloxy-propyl)-1-methyl-1H-imidazole (DPIM) N,N,N′,N′-tetramethyl-N,N′-bis(2-hydroxyethyl)-2,3-dioleoyloxy-1,4-butane-diammonium iodide) (Tfx-50); N-1-(2,3-dioleoyloxy) propyl-N,N,N-trimethyl ammonium chloride (DOTMA) or other N—(N,N-1-dialkoxy)-alkyl-N,N,N-trisubstituted ammonium surfactants; 1,2 dioleoyl-3-(4′-trimethylammonio) butanol-sn-glycerol (DOBT) or cholesteryl (4′ trimethylammonia) butanoate (ChOTB) where the trimethylammonium group is connected via a butanol spacer arm to either the double chain (for DOTB) or cholesteryl group (for ChOTB); DORI (DL-1,2-dioleoyl-3-dimethylaminopropyl-.beta.-hydroxyethylammonium) or DORIE (DL-1,2-0-dioleoyl-3-dimethylaminopropyl-.beta.-hydroxyethylammoniu- -m) (DORIE) or analogs thereof as disclosed in WO 93/03709; 1,2-dioleoyl-3-succinyl-sn-glycerol choline ester (DOSC); cholesteryl hemisuccinate ester (ChOSC); lipopolyamines such as dioctadecylamidoglycylspermine (DOGS) and dipalmitoyl phosphatidylethanolamylspermine (DPPES), cholesteryl-3 β-carboxyl-amido-ethylenetrimethylammonium iodide, 1-dimethylamino-3-trimethylammonio-DL-2-propyl-cholesteryl carboxylate iodide, cholesteryl-3-O-carboxyamidoethyleneamine, cholesteryl-3-.beta.-oxysuccinamido-ethylenetrimethylammonium iodide, 1-dimethylamino-3-trimethylammonio-DL-2-propyl-cho lesteryl-3-β-oxysu-ccinate iodide, 2-(2-trimethylammonio)-ethylmethylamino ethyl-cho lesteryl-3-.beta.-oxysuccinate iodide, 3-β-N—(N′,N′-dimethylaminoethane) carbamoyl cholesterol (DC-chol), and 3-β-N-(polyethyleneimine)-carbamoylcholesterol; 0,0′-dimyristyl-N-lysyl aspartate (DMKE); 0,0′-dimyristyl-N-lysyl-glutamate (DMKD); 1,2-dimyristyloxypropyl-3-dimethyl-hydroxy ethyl ammonium bromide (DMRIE); 1,2-dilauroyl-sn-glycero-3-ethylphosphocholine (DLEPC); 1,2-dimyristoyl-sn-glycero-3-ethylphosphocholine (DMEPC); 1,2-dioleoyl-sn-glycero-3-ethylphosphocholine (DOEPC); 1,2-dipalmitoyl-sn-glycero-3-ethylphosphocholine (DPEPC); 1,2-distearoyl-sn-glycero-3-ethylphosphocholine (DSEPC); 1,2-dioleoyl-3-trimethylammonium propane (DOTAP); dioleoyl dimethylaminopropane (DODAP); 1,2-palmitoyl-3-trimethylammonium propane (DPTAP); 1,2-distearoyl-3-trimethylammonium propane (DSTAP), 1,2-myristoyl-3-trimethylammonium propane (DMTAP); and sodium dodecyl sulfate (SDS). Furthermore, structural variants and derivatives of the any of the described cationic lipids are also contemplated.

In some aspects, the cationic lipid is selected from the group consisting of DOTAP, DOTMA, DOEPC, and combinations thereof. In other aspects, the cationic lipid is DOTAP. In yet other aspects, the cationic lipid is DOTMA. In other aspects, the cationic lipid is DOEPC. In some aspects, the cationic lipid is purified.

In some embodiments, the cationic lipid is an enantiomer of a cationic lipid. The term “enantiomer” refers to a stereoisomer of a cationic lipid which is a non-superimposable mirror image of its counterpart stereoisomer, for example R and S enantiomers. In various examples, the enantiomer is R-DOTAP or S-DOTAP. In one example, the enantiomer is R-DOTAP. In another example, the enantiomer is S-DOTAP. In some aspects, the enantiomer is purified.

In one aspect, the cationic lipid is selected from the group consisting of DOTAP, DDA, DOEPC, DOTMA, R-DOTAP, R-DDA, R-DOEPC, R-DOTMA, S-DOTAP, S-DDA, S-DOEPC, S-DOTMA, variations thereof and analogs thereof.

In another aspect, the multiepitope peptide is oxidized, cross-linked, pegylated, glycosylated, phosphorylated, palmitoylated, methylated, or biotinylated.

In one aspect, the epitope peptides are mucin 1 (MUC1) epitopes.

In one aspect, the MUC1 epitopes have MHC affinity for HLA-A2, A3, A11 and A24.

In another aspect, the MUC1 epitopes are recognized by a CD4+ T cell receptor and/or by a CD8+ T cell receptor.

In one aspect, the one or more epitope peptides include a cleavable anionic N-terminal extension. In some aspects, the cleavable anionic N-terminal extension includes the amino acid sequence SSEEDE (SEQ ID NO:38) or SSEEDEE (SEQ ID NO:39).

In another aspect, the one or more multiepitope peptides are encapsulated in liposomes including cationic lipids or mixed as micelles with preformed cationic lipid nanoparticles. In some aspects, the one or more multiepitope peptides and the preformed cationic lipid nanoparticles are mixed at a 1:1 ratio.

In one aspect, the composition further includes an enhancer agonist epitope and/or an analog thereof.

In an additional embodiment, the invention provides a vaccine composition including: (a) one or more multiepitope peptides including at least two epitope peptides recognized by CD4⁺ and/or CD8⁺ T cell receptors, wherein the epitope peptides are mucin 1 (MUC1) epitopes; and (b) a cationic lipid.

According to the invention, the term “vaccine” relates to a pharmaceutical preparation (pharmaceutical composition) or product that upon administration induces an immune response, in particular a cellular immune response, which recognizes and attacks a pathogen or a diseased cell such as a cancer cell. A vaccine may be used for the prevention or treatment of a disease. The term “individualized cancer vaccine” concerns a particular cancer patient and means that a cancer vaccine is adapted to the needs or special circumstances of an individual cancer patient.

In one aspect, the cationic lipid is selected from the group consisting of DOTAP, DDA, DOEPC, DOTMA, R-DOTAP, R-DDA, R-DOEPC, R-DOTMA, S-DOTAP, S-DDA, S-DOEPC, S-DOTMA, variations thereof and analogs thereof. In some aspects, the cationic lipid is R-DOTAP.

In another aspect, the at least two epitope peptides recognized by CD4+ and/or CD8+ T cell receptors include a sequence including at least 80% identity to the amino acid sequence of any one of SEQ ID NOs:2-37 and 42-43. In one aspect, the MUC1 epitopes have MHC affinity for HLA-A2, A3, A11 and A24. In another aspect, the one or more epitope peptides are oxidized, cross-linked, pegylated, glycosylated, phosphorylated, palmitoylated, methylated, or biotinylated. In some aspects, the one or more epitope peptides are palmitoylated. In one aspect, the one or more epitope peptides include a cleavable anionic N-terminal extension. In some aspects, the cleavable anionic N-terminal extension includes the amino acid sequence SSEEDE (SEQ ID NO:38) or SSEEDEE (SEQ ID NO:39). In another aspect, the at least two epitope peptides recognized by CD4+ and/or CD8+ T cell receptors include a sequence including at least 80% identity to the amino acid sequence of any one of SEQ ID NOs:9, 10, 13, 14, 18, 19, 42 or 43. In one aspect, the multiepitope peptide includes a sequence including at least 80%, 85%, 90% or 95% sequence identity to SEQ ID NO: 1. In another aspect, the at least two epitope peptides recognized by CD4+ and/or CD8+ T cell receptors are covalently modified to improve association with a cationic lipid. In some aspect, the covalent modification includes palmitoylation or addition of an anionic sequence. In various aspect, the modified peptides include an amino acid sequence comprising at least 80% identity to SEQ ID NOs: 18, 19, 42 or 43. In another aspect, the one or more multiepitope peptides are encapsulated in liposomes including cationic lipids or mixed as micelles with preformed cationic lipid nanoparticles. In some aspects, the at least two multiepitope peptides and the preformed cationic lipid nanoparticles are mixed at a 1:1 ratio. In one aspect, the one or more multiepitope peptides comprise a sequence comprising at least 80% identity to the amino acid sequence of SEQ ID NO:1.

In a further embodiment, the invention provides a method of treating cancer in a subject including administering to the subject a vaccine composition including: (a) a multiepitope peptide including one or more mucin 1 (MUC1) derived epitopes; and (b) a cationic lipid, thereby treating cancer in the subject.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treating cancer).

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization.

In one aspect, the one or more MUC1 derived epitopes include a sequence comprising at least 80% identity to the amino acid sequence of any one of SEQ ID NOs:2-37 and 42-43. In another aspect, the multiepitope peptide includes a sequence including at least 80%, 85%, 90% or 95% identity to the amino acid sequence of SEQ ID NO:1.

In one aspect, the cationic lipid is selected from the group consisting of DOTAP, DDA, DOEPC, DOTMA, R-DOTAP, R-DDA, R-DOEPC, R-DOTMA, S-DOTAP, S-DDA, S-DOEPC, S-DOTMA, variations thereof and analogs thereof. In some aspects, the cationic lipid is R-DOTAP.

In another aspect, the multiepitope peptide is encapsulated in cationic lipid nanoparticles or mixed as micelles with preformed cationic lipid nanoparticles.

In some aspects, the multiepitope peptide and the preformed cationic lipid nanoparticles are mixed at a 1:1 ratio.

In one aspect, the one or more MUC1 derived epitopes induce the presentation of non-HLA restricted peptides to CD4⁺ and CD8⁺ T cells by antigen presenting cells.

The epitopes and multiepitope peptides described herein are HLA-class I and/or HLA-class II non-restricted epitopes.

The human leukocyte antigen (HLA) system or complex is a complex of genes located on chromosome 6 in humans, and which encode cell-surface proteins responsible for the regulation of the immune system. The HLA system is also known as the human version of the major histocompatibility complex (MHC) found in many animals. HLA genes are highly polymorphic, which means that they have many different alleles, allowing them to fine-tune the adaptive immune system. HLAs corresponding to MHC class I (A, B, and C), all of which are the HLA Class1 group, present peptides from inside the cell. These peptides are produced from digested proteins that are broken down in the proteasomes. In general, these particular peptides are small polymers, of about 8-10 amino acids in length. Foreign antigens presented by MHC class I attract T-lymphocytes called killer T-cells (also referred to as CD8-positive or cytotoxic T-cells) that destroy cells. MHC class I proteins associate with B2-microglobulin, which unlike the HLA proteins is encoded by a gene on chromosome 15. HLAs corresponding to MHC class II (DP, DM, DO, DQ, and DR) present antigens from outside of the cell to T-lymphocytes. These particular antigens stimulate the multiplication of T-helper cells (also called CD4-positive T cells), which in turn stimulate antibody-producing B-cells to produce antibodies to that specific antigen. Self-antigens are suppressed by regulatory T cells.

MHC-restricted antigen recognition, MHC restriction or HLA-restriction, refers to the fact that a T cell can interact with a self-major histocompatibility complex molecule and a foreign peptide bound to it but will only respond to the antigen when it is bound to a particular MHC molecule. When foreign proteins enter a cell, they are broken into peptides. These peptides or antigens can derive from pathogens such as viruses or intracellular bacteria. Foreign peptides are brought to the surface of the cell and presented to T cells by proteins called the major histocompatibility complex (MHC). During T cell development, T cells go through a selection process in the thymus to ensure that the T cell receptor (TCR) will not recognize MHC molecule presenting self-antigens, i.e., that its affinity is not too high. High affinity means it will be autoreactive, but no affinity means it will not bind strongly enough to the MHC. The selection process results in developed T cells with specific TCRs that might only respond to certain MHC molecules but not others. The fact that the TCR will recognize only some MHC molecules but not others contribute to “MHC restriction”. The biological reason of MHC restriction is to prevent supernumerary wandering lymphocytes generation, hence energy saving and economy of cell-building materials. T-cells are a type of lymphocyte that is significant in the immune system to activate other immune cells. T-cells will recognize foreign peptides through T-cell receptors (TCRs) on the surface of the T cells, and then perform different roles depending on the type of T cell they are in order to defend the host from the foreign peptide, which may have come from pathogens like bacteria, viruses or parasites. Enforcing the restriction that T cells are activated by peptide antigens only when the antigens are bound to self-MHC molecules, MHC restriction adds another dimension to the specificity of T cell receptors so that an antigen is recognized only as peptide-MHC complexes. MHC restriction in T cells occurs during their development in the thymus, specifically positive selection. Only the thymocytes (developing T cells in the thymus) that are capable of binding, with an appropriate affinity, with the MHC molecules can receive a survival signal and go on to the next level of selection. MHC restriction is significant for T cells to function properly when it leaves the thymus because it allows T cell receptors to bind to MHC and detect cells that are infected by intracellular pathogens, viral proteins and bearing genetic defects.

In another aspect, treating cancer includes preventing progression of cancer in the subject.

Cancer is a group of diseases involving abnormal cell growth with the potential to invade or spread to other parts of the body. In 2015, about 90.5 million people had cancer, about 14.1 million new cases occur a year and it caused about 8.8 million deaths (15.7% of deaths). The most common types of cancer in males are lung cancer, prostate cancer, colorectal cancer and stomach cancer. In females, the most common types are breast cancer, colorectal cancer, lung cancer and cervical cancer.

The term “cancer” refers to a group of diseases characterized by abnormal and uncontrolled cell proliferation starting at one site (primary site) with the potential to invade and to spread to other sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof.

As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, pancreatic, brain, and head and neck cancers, melanoma, sarcoma, multiple myeloma, leukemia, lymphoma, and the like.

Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood’, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma a Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

In one aspect, the cancer includes MUC1 expressing cancer cells.

MUC1 is expressed in a variety of tissues and organ, including nasopharynx, bronchus, stomach, colon, rectum, gallbladder, fallopian tube, endometrium, cervix, placenta, lung, esophagus, duodenum, small intestine, pancreas, kidney, urinary bladder, testis, epididymis, seminal vesicle, breast, appendix, adrenal gland, oral mucosa, salivary gland, prostate, skin, lymph node, tonsil and bone marrow. Accordingly, non-limiting examples of cancer including MUC1 expressing cells include nasopharyngeal carcinoma, lung cancer, stomach cancer, colorectal cancer, gallbladder cancer, tubal cancer, endometrial cancer, cervical cancer, esophageal cancer, duodenal cancer, small intestine cancer, pancreatic cancer, renal cancer, bladder cancer, testicular cancer, epididymal, seminal vesicle cancer, breast cancer, adrenal cancer, buccal cancer, salivary gland cancer, prostate cancer, skin cancer, melanoma, lymphoma, tonsil cancer, myeloma and multiple myeloma.

In some aspects, the cancer is prostate cancer, breast cancer or acute myeloid leukemia (AML).

In another aspect, the method further includes administering to the subject an anti-cancer treatment.

As used herein, the term “anti-cancer treatment” is meant to refer to any therapeutic approach that can be used for the treatment of cancer. It includes, but is not limited to surgery, radiotherapy, chemotherapy, immunotherapy, targeted therapy, and any combination thereof.

In some aspects administration can be in combination with one or more additional therapeutic agents. The phrases “combination therapy”, “combined with” and the like refer to the use of more than one medication or treatment simultaneously to increase the response. The compositions of the present invention might for example be used in combination with other drugs or treatment in use to treat cancer. Specifically, the administration of the peptides, multiepitope peptides, or vaccines described herein to a subject can be in combination with another anti-cancer therapy such as an immune checkpoint inhibitor therapy. Such therapies can be administered prior to, simultaneously with, or following administration of the composition of the present invention.

In some aspects, the anti-cancer treatment includes an immune checkpoint inhibitor therapy.

“Checkpoint inhibitor therapy” is a form of cancer treatment currently that uses immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligand, PD-1 ligand 1 (PD-L1, CD274), cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), KIR (Killer-cell Immunoglobulin-like Receptor), LAG3 (Lymphocyte Activation Gene-3), TIM-3 (T-cell Immunoglobulin domain and Mucin domain 3), and VISTA (V-domain Ig suppressor of T cell activation).

Programmed cell death protein 1, also known as PD-1 and CD279 (cluster of differentiation 279), is a cell surface receptor that plays an important role in down-regulating the immune system and promoting self-tolerance by suppressing T cell inflammatory activity. PD-1 is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells).

PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-L1 is expressed on almost all murine tumor cell lines, including PA1 myeloma, P815 mastocytoma, and B16 melanoma upon treatment with IFN-γ. PD-L2 expression is more restricted and is expressed mainly by DCs and a few tumor lines.

CTLA4 or CTLA-4 (cytotoxic T-lymphocyte-associated protein 4), also known as CD152 (cluster of differentiation 152), is a protein receptor that, functioning as an immune checkpoint, downregulates immune responses. CTLA4 is constitutively expressed in regulatory T cells but only upregulated in conventional T cells after activation—a phenomenon which is particularly notable in cancers. CTLA4 is a member of the immunoglobulin superfamily that is expressed by activated T cells and transmits an inhibitory signal to T cells. CTLA4 is homologous to the T-cell co-stimulatory protein, CD28, and both molecules bind to CD80 and CD86, also called B7-1 and B7-2 respectively, on antigen-presenting cells. CTLA-4 binds CD80 and CD86 with greater affinity and avidity than CD28 thus enabling it to outcompete CD28 for its ligands. CTLA4 transmits an inhibitory signal to T cells, whereas CD28 transmits a stimulatory signal. CTLA4 is also found in regulatory T cells and contributes to its inhibitory function. T cell activation through the T cell receptor and CD28 leads to increased expression of CTLA-4.

There are several checkpoint inhibitors that are currently used to treat cancer. PD-1 inhibitors include Pembrolizumab (Keytruda) and Nivolumab (Opdivo). PD-L1 inhibitors include Atezolizumab (Tecentriq), Avelumab (Bavencio) and Durvalumab (Imfinzi). CTLA-4 inhibitors include Iplimumab (Yervoy). There are several other checkpoint inhibitors being developed including an anti B7-H3 antibody (MGA271), an anti-KIR antibody (Lirilumab) and an anti-LAG3 antibody (BMS-986016).

By “targeted therapy”, it is meant any molecularly targeted therapy used to block the growth of cancer cells by interfering with specific targeted molecules needed for carcinogenesis and tumor growth, rather than by simply interfering with all rapidly dividing cells (e.g., with traditional chemotherapy). Biomarkers are usually required to aid the selection of patients who will likely respond to a given targeted therapy. Non-limiting examples of targeted therapy include: tyrosine-kinase inhibitors, small molecules drug conjugates, serine/threonine kinase inhibitors, monoclonal antibodies and histone deacetylase (HDAC) inhibitor.

In other aspects, the targeted therapy comprises a HDAC inhibitor.

Histone deacetylases (HDAC) are a class of enzymes that remove acetyl groups (O═C—CH3) from an ε-N-acetyl lysine amino acid on both histone and non-histone proteins. HDACs allow histones to wrap the DNA more tightly. This is important because DNA is wrapped around histones, and DNA expression is regulated by acetylation and de-acetylation. HDAC's action is opposite to that of histone acetyltransferase. HDAC proteins are now also called lysine deacetylases (KDAC), to describe their function rather than their target, which also includes non-histone proteins. In general, they suppress gene expression.

Histone deacetylase inhibitors (HDAC inhibitors, HDACi, HDIs) are chemical compounds that inhibit histone deacetylases. The “classical” HDIs act exclusively on Class I, II and Class IV HDACs by binding to the zinc-containing catalytic domain of the HDACs. These classical HDIs can be classified into several groupings named according to the chemical moiety that binds to the zinc ion (except cyclic tetrapeptides which bind to the zinc ion with a thiol group). Some examples in decreasing order of the typical zinc binding affinity: 1. hydroxamic acids (or hydroxamates), such as trichostatin A, 2. cyclic tetrapeptides (such as trapoxin B), and the depsipeptides, 3. benzamides, 4. electrophilic ketones, and 5. the aliphatic acid compounds such as phenylbutyrate and valproic acid.

“Second-generation” HDIs include the hydroxamic acids vorinostat (SAHA), belinostat (PXD101), LAQ824, and panobinostat (LBH589); and the benzamides: entinostat (MS-275), tacedinaline (CI994), and mocetinostat (MGCD0103). The sirtuin Class III HDACs are dependent on NAD+ and are, therefore, inhibited by nicotinamide, as well as derivatives of NAD, dihydrocoumarin, naphthopyranone, and 2-hydroxynaphthaldehydes. Non-limiting examples of HDAC inhibitor include: Istodax (Romidepsin), Zolinza (Vorinostat), Farydak (Panobinostat) and Belodaq (Belinostat).

In one aspect, treating cancer includes inducing a MUC1-specific polyfunctional cytolytic T cell response in the subject.

The term “immune response” refers to an integrated bodily response to an antigen and preferably refers to a cellular immune response or a cellular as well as a humoral immune response. The immune response may be protective/preventive/prophylactic and/or therapeutic.

The immune system is a system of biological structures and processes within an organism that protects against disease. This system is a diffuse, complex network of interacting cells, cell products, and cell-forming tissues that protects the body from pathogens and other foreign substances, destroys infected and malignant cells, and removes cellular debris: the system includes the thymus, spleen, lymph nodes and lymph tissue, stem cells, white blood cells, antibodies, and lymphokines. B cells or B lymphocytes are a type of lymphocyte in the humoral immunity of the adaptive immune system and are important for immune surveillance. T cells or T lymphocytes are a type of lymphocyte that plays a central role in cell-mediated immunity. There are two major subtypes of T cells: the killer T cell and the helper T cell. In addition, there are suppressor T cells which have a role in modulating immune response. Killer T cells only recognize antigens coupled to Class I MHC molecules, while helper T cells only recognize antigens coupled to Class II MHC molecules. These two mechanisms of antigen presentation reflect the different roles of the two types of T cell. A third minor subtype are the γδ T cells that recognize intact antigens that are not bound to MHC receptors. In contrast, the B cell antigen-specific receptor is an antibody molecule on the B cell surface and recognizes whole pathogens without any need for antigen processing. Each lineage of B cell expresses a different antibody, so the complete set of B cell antigen receptors represent all the antibodies that the body can manufacture.

A “cellular immune response”, a “cellular response”, a “cellular response against an antigen” or a similar term is meant to include a cellular response directed to cells characterized by presentation of an antigen with class I or class II MHC. The cellular response relates to cells called T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells. In preferred embodiments, the present invention involves the stimulation of an anti-tumor CTL response against tumor cells expressing one or more tumor expressed antigens and preferably presenting such tumor expressed antigens with class I MHC.

The terms “immunoreactive cell” “immune cells” or “immune effector cells” in the context of the present invention relate to a cell which exerts effector functions during an immune reaction. An “immunoreactive cell” preferably is capable of binding an antigen or a cell characterized by presentation of an antigen, or an antigen peptide derived from an antigen and mediating an immune response. For example, such cells secrete cytokines and/or chemokines, secrete antibodies, recognize cancerous cells, and optionally eliminate such cells. For example, immunoreactive cells comprise T cells (cytotoxic T cells, helper T cells, tumor infiltrating T cells), B cells, natural killer cells, neutrophils, macrophages, and dendritic cells.

“Inducing an immune response” may mean that there was no immune response against a particular antigen before induction, but it may also mean that there was a certain level of immune response against a particular antigen before induction and after induction said immune response is enhanced. Thus, “inducing an immune response” also includes “enhancing an immune response”. Preferably, after inducing an immune response in a subject, said subject is protected from developing a disease such as a cancer disease or the disease condition is ameliorated by inducing an immune response. For example, an immune response against a tumor expressed antigen may be induced in a patient having a cancer disease or in a subject being at risk of developing a cancer disease. Inducing an immune response in this case may mean that the disease condition of the subject is ameliorated, that the subject does not develop metastases, or that the subject being at risk of developing a cancer disease does not develop a cancer disease.

Within the human mucin 1 oncoprotein multiple high affinity MHC-binding peptide antigens specific for HLA-A2, A3, A11 and A24 have been identified (Table 1). MHC-binding peptide antigens specific for HLA-A2 have been identified: YLAIVYLIAL (SEQ ID NO:9, C1A), YLIALAVCQV (SEQ ID NO:10, C2A), YLAPPAHGV (SEQ ID NO:13, VIA) and YLDTRPAPV (SEQ ID NO:14, V2A).

TABLE 1

| | | | HLA |
|---|---|---|---|
| Identifier | Agonist Sequence | Natural sequence | Binding |
| C1A | YLAIVTLIAL (SEQ ID NO: 20) | ALAIVYLIAL (SEQ ID NO: 29) | A2 |
| C2A | YLIALAVCQV (SEQ ID NO: 21) | YLIALAVCQC (SEQ ID NO: 30) | A2 |
| V1A | YLAPPAHGV (SEQ ID NO: 22) | STAPPAHGV (SEQ ID NO: 31) | A2 |
| V2A | YLDTRPAPV (SEQ ID NO: 23) | APDTRPAPG (SEQ ID NO: 32) | A2 |
| C3A | YLSYTNPAV (SEQ ID NO: 24) | SLSYTNPAV (SEQ ID NO: 33) | A2 |
| C4A | ALFIVYLIAK (SEQ ID NO: 25) | ALAIVYLIAL (SEQ ID NO: 34) | A3 |
| C5A | SLFRSPYEK (SEQ ID NO: 26) | STDRSPYEK (SEQ ID NO: 35) | A3 |
| C6A | KYHPMSEYAL (SEQ ID NO: 27) | TYHPMSEYPT (SEQ ID NO: 36) | A24 |
| C7A | KYTNPAVAL (SEQ ID NO: 28) | SYTNPAVAA (SEQ ID NO: 37) | A24 |

The native and modified agonist peptide antigen sequences derived from human oncoprotein MUC1 are shown, with amino acid substitutions indicated in bold-underline showing the modifications utilized to create agonist antigens with enhanced MHC binding affinities.

Specifically, all four peptides C1A (SEQ ID NO:9) and C2A (SEQ ID NO:10) VIA (SEQ ID NO:13) and V2A (SEQ ID NO:14) have been modified by amino acid substitutions in their natural sequences to increase MHC binding affinity, thus increasing their cellular display on antigen-presenting human dendritic cells and their potential effectiveness in inducing high levels of antigen-specific cytotoxic T-cells (See Table 1). The agonist amino acid modifications, however, have the negative consequence of resulting in the already hydrophobic peptides C1A and C2A having no polar residues to promote aqueous solubility. These highly hydrophobic peptide antigens with very high MHC binding but limited aqueous solubility are therefore ineffective molecules in vivo for use in traditional vaccine compositions due to the difficulty in successfully synthesizing the antigens at high purity and delivering the antigens to antigen presenting cells of the immune system in standard vaccine compositions such as CFA or other oil in water emulsions or when combined with any aqueous compatible formulation. Three successful approaches to solve this problem were developed:

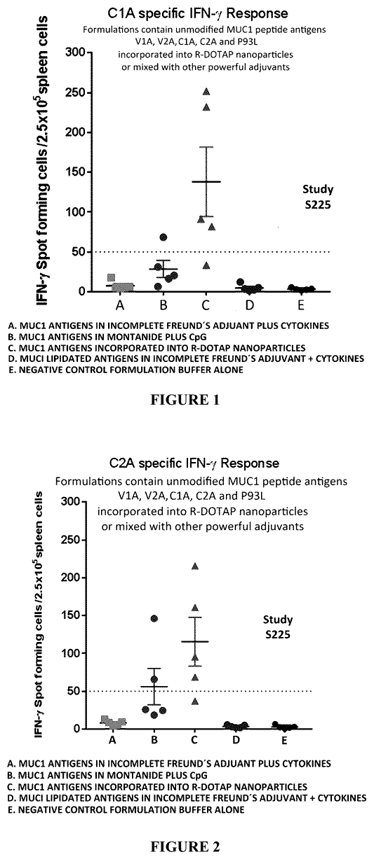

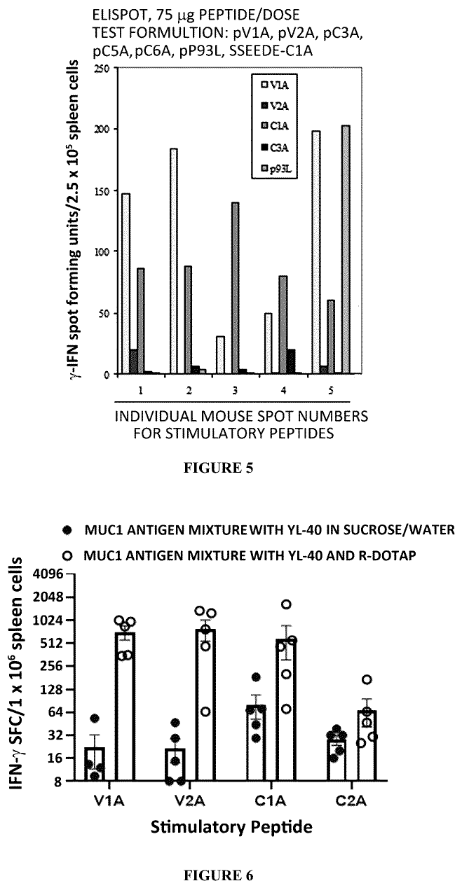

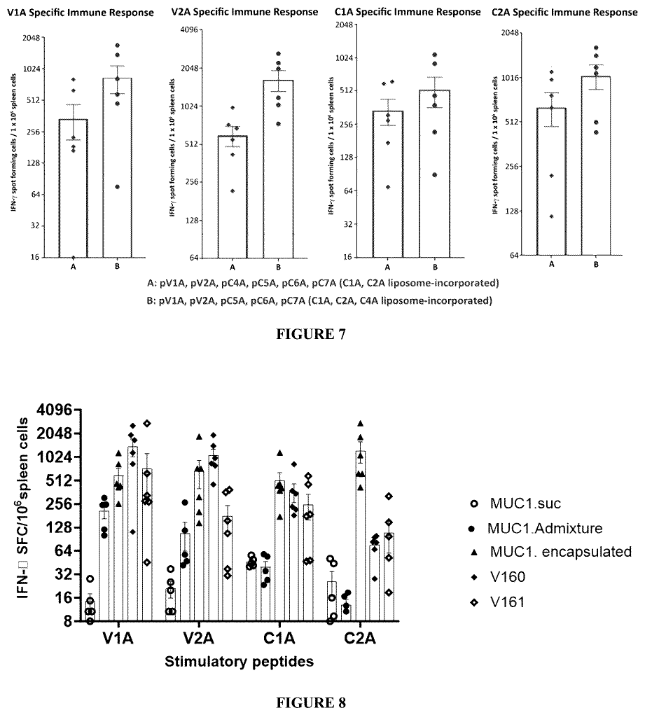

1. By synthesizing these hydrophobic peptides with an anionic charged cleavable N-terminal extension on the peptide antigens which both allows the peptides to be synthesized at high purity and provides peptide solubility and compatibility with cationic R-DOTAP immunostimulatory nanoparticles. The association of the anionic domains of the antigens with the cationic surface of the nanoparticles results in high levels of antigen delivery and immune cell activation (FIGS. 4 and 5).
2. By delivering these two highly hydrophobic antigens by inserting them into the nonpolar interior membrane bilayer of cationic surface charged liposomal nanoparticles formed from the unique enantiomeric immunostimulatory lipid R-DOTAP. This unique delivery vehicle effectively traps the water-insoluble peptide antigens and delivers them quantitatively in the highly charged water-compatible immunostimulatory nanoparticles to dendritic cells of the immune system where the antigens are displayed on the surface MHC of the activated dendritic cells to induce high levels of antigen-specific cytotoxic T-cells (FIGS. 1, 2 and 3). This entrapment and delivery of the nonpolar peptide antigens in the enantiomeric cationic lipid nanoparticle lipid bilayer results in very high levels of induction of antigen specific T-cells in murine humanized HLA-A2 transgenic mouse ELISpot assays (FIGS. 1, 2 and 3).
3. A third approach to resolve the problem of delivery of hydrophobic peptide antigens C1A and C2A has also been developed involving the incorporation of the amino acid sequences into a much larger peptide derived from the sequences of the MUC 1 oncoprotein that can be effectively associated with and delivered with R-DOTAP immunostimulatory nanoparticles in an aqueous environment. This large peptide, YL40 (SEQ ID NO:1) contains both the hydrophobic agonist peptides on the N-terminus of the molecule where they can be effectively processed by intracellular proteases in dendritic cells to yield the individual agonist peptide antigens for display on cell surface MHC. This approach has been found to yield high level detection of peptide agonist C1A specific T-cell responses after humanized transgenic HLA-A mouse vaccination and ELISpot analysis (FIGS. 6 and 7).

In the design of peptide-based immunotherapeutics designed to elicit cytotoxic T cells capable of recognizing and killing tumor cells expressing target antigen, the peptide antigens must be delivered in association with potent immunostimulation capable of promoting effective antigen uptake by dendritic cells, directing peptide antigen processing into the class I (CD8) pathway, and proving correct cytokine activation and signaling to induce large numbers of multiple cytokine producing effector T-cells. The inventors herein have identified peptide sequences that uniquely and effectively associate with R-DOTAP immunostimulatory nanoparticles, which are taken up and processed in this context by dendritic cells and generate high levels of multi-cytokine producing MUC1-specific killer CD8 T-cells. Numerous other combinations of MUC1 peptide sequences including short specific peptide antigens and other long overlapping peptide sequences from the MUC1 protein have been demonstrated to be ineffective when combined with or incorporated into R-DOTAP immunostimulatory nanoparticles (see Example 5 below). Thus, the unique combination of short lipidated agonist peptide epitopes which form high molecular weight micellar structures and long MUC1 peptide sequences reported here results in strong antigen-specific immune responses when delivered in association with R-DOTAP immunostimulatory nanoparticles. The peptides may be incorporated into immunogenic compositions, such as vaccines. The peptide sequences utilized for the resulting compositions have the benefit of inducing powerful cytotoxic T cell responses.

Disclosed herein are methods for the design and use of unique peptide sequences comprising human cytotoxic T lymphocyte (CTL) epitopes encoded in MUC1 protein. The sequences shown in Table 1, include YL40 (SEQ ID NO:1), pV1A (SEQ ID NO:2), pV2A (SEQ ID NO:3), pC5A (SEQ ID NO:4), pC6A (SEQ ID NO:5), pC7A (SEQ ID NO:6), are unique epitope enhanced peptides selected to facilitate the processing and presentation of T cell antigens encoded in the MUC1 protein when delivered in association with R-DOTAP nanoparticles.

TABLE 1
Peptide Sequences:

| | | | HLA | |
|---|---|---|---|---|
| ID | Modification | Sequence | affinity | Code |
| SEQ ID | None | YLAIVYLIALAVCQVRRKNYGQLDIFP | A2, A24 | YL40 |
| NO: 1 | | ARDKYHPMSEYAL | | |
| SEQ ID | Epsilon- | KSS-YLAPPAHGV | A2 | pVIA |
| NO: 2 | palmitoyl | | | |
| SEQ ID | Epsilon- | KSS-YLDTRPAPV | A2 | pV2A |
| NO: 3 | palmitoyl | | | |
| SEQ ID | Epsilon- | KSS-SLFRSPYEK | A24 | pC5A |
| NO: 4 | palmitoyl | | | |
| SEQ ID | Epsilon- | KSS-KYHPMSEYAL | A3 | pC6A |
| NO: 5 | palmitoyl | | | |
| SEQ ID | Epsilon- | KSS-KYTNPAVAL | A24 | pC7A |
| NO: 6 | palmitoyl | | | |
| SEQ ID | None | YLAIVYLIAL | A2 | CIA |
| NO: 9 | | | | |
| SEQ ID | None | YLIALAVCQV | A2 | C2A |
| NO: 10 | | | | |
| SEQ ID | None | ALWGQDVTSV | A2 | P93L |
| NO: 11 | | | | |
| SEQ ID | None | YLSYTNPAV | A2 | C3A |
| NO: 12 | | | | |
| SEQ ID | None | YLAPPAHGV | A2 | VIA |
| NO: 13 | | | | |
| SEQ ID | None | YLDTRPAPV | A2 | V2A |
| NO: 14 | | | | |
| SEQ ID | Epsilon- | KSS-YLSYTNPAV | A2 | pC3A |
| NO: 15 | palmitoyl | | | |
| SEQ ID | Epsilon- | KSS-ALWGQDVTSV | A2 | P93L |
| NO: 16 | palmitoyl | | | |
| SEQ ID | Epsilon- | KSS-ALFIVYLIAK | A3 | pC4A |
| NO: 17 | palmitoyl | | | |
| SEQ ID | | SSEEDE-YLAIVYLIAL | A2 | sCIA |
| NO: 18 | | | | |
| SEQ ID | | SSEEDE-YLIALAVCQV | A2 | sC2A |
| NO: 19 | | | | |
| SEQ ID | | YLIALAVCQVRRKNYGQLDIFPARDKY | A2, A24 | YL35 |
| NO: 40 | | HPMSEYAL- | | |
| SEQ ID | | STDRSPYEKVSAGNGGSSLSYTNPAVA | A3, A24 | SL33 |
| NO: 41 | | ATSANL | | |
| SEQ ID | | SSEEDEE-YLAIVYLIAL | A2 | sCIA |
| NO: 42 | | | | |
| SEQ ID | | SSEEDEE-YLIALAVCQV | A2 | SC2A |
| NO: 43 | | | | |

### EXAMPLES

### Example 1

### Design of MUC1 Protein Derived Peptides

Disclosed herein are methods for the design and use of unique peptide sequences (SEQ ID NOs: 1-6) derived from MUC1 protein designed to be effectively processed and presented to T cells when delivered in association with R-DOTAP immunostimulatory nanoparticles.

Here, the limited aqueous solubility of the highly hydrophobic peptides C1A and C2A was overcome by developing three successful approaches to solve this problem: (1) synthesizing these hydrophobic peptides with an anionic charged cleavable N-terminal extension on the peptide antigens, (2) by delivering these two highly hydrophobic antigens by inserting them into the nonpolar interior membrane bilayer of cationic surface charged liposomal nanoparticles formed from the unique enantiomeric immunostimulatory lipid R-DOTAP, and (3) by incorporating the amino acid sequences of individual epitope peptides into a larger (multiepitope) peptide.

### Example 2

### Effect of the Addition of an Anionic Charged Cleavable N-Terminal Extension

Synthesizing these hydrophobic peptides with an anionic charged cleavable N-terminal extension on the peptide antigens both allowed the peptides to be synthesized at high purity and provided peptide solubility and compatibility with cationic R-DOTAP immunostimulatory nanoparticles. The association of the anionic domains of the antigens with the cationic surface of the nanoparticles resulted in high levels of antigen delivery and immune cell activation (FIGS. 4 and 5).

As illustrated in FIG. 4 HLA-A2 specific CD8 T cell responses to formulations including peptide antigen SSEEDE-C1A (SEQ ID NO:18) and SSEEDE-C2A (SEQ ID NO:19) formulated with R-DOTAP nanoparticles and a micellar mixture with six lipidated peptide agonist antigens, pC3A: (SEQ ID NO:15), pV1A: (SEQ ID NO:2), pV2A: (SEQ ID NO:3), pC5A: (SEQ ID NO:4), pC6A: (SEQ ID NO:5), pP93L: (SEQ ID NO: 16) were evaluated. Highly hydrophobic peptide antigens C1A and C2A are ordinarily inactive in generating immune responses when included in antigen mixtures mixed with R-DOTAP immunostimulatory nanoparticles due to low peptide solubility in aqueous systems. Addition of an anionic amino acid sequence (SSEEDE, SEQ ID NO:38) to the N-terminus of the hydrophobic peptides both allowed synthesis of high purity antigen and ensured effective delivery of the otherwise insoluble antigens to dendritic cells of the immune system through strong interactions with the cationic nanoparticles in suspension. These results were confirmed for four replicate preparations of the antigen mixture/R-DOTAP formulations. The efficacy of these vaccine formulations in generating T cell responses was measured by dosing HLA-A2 expressing transgenic mice (AAD mice) with two 0.1 ml injections of the vaccine delivered subcutaneously on day 0 and day 7. Negative control groups mice were vaccinated with MUC1 peptides formulated in sucrose buffer. Immune responses specific to vaccine formulation were measured in an ELISPOT assay by enumerating MUC1 antigen-specific T cells, in triplicate wells, in the spleens obtained from vaccinated mice. Each bar in the graph represents the mean SFU per 250,000 splenocytes for a group of vaccinated mice. Error bars represent mean±SEM for five mice in each group. For identifying MUC1-specific T cells, splenocytes were stimulated with peptide sequences C1A (SEQ ID NO:9: YLAIVYLIAL), C2A (SEQ ID NO:10), V1A (SEQ ID NO:13), V2A (SEQ ID NO:14:) C3A (SEQ ID NO:12) and p93L (SEQ ID NO:11)

As shown in FIG. 5 HLA-A2 specific CD8 T cell responses to formulations including peptide antigen SSEEDE-C1A (SEQ ID NO:9) and SSEEDE-C2A (SEQ ID NO:10) formulated with R-DOTAP nanoparticles and a micellar mixture with six lipidated peptide agonist antigens, pC3A: (SEQ ID NO:15), pV1A: (SEQ ID NO:2), pV2A: (SEQ ID NO:3), pC5A: (SEQ ID NO:4), pC6A: (SEQ ID NO:5), pP93L: (SEQ ID NO:16) were assessed. Highly hydrophobic peptide antigens C1A and C2A are ordinarily inactive in generating immune responses when included in antigen mixtures mixed with R-DOTAP immunostimulatory nanoparticles due to low peptide solubility in aqueous systems. Addition of an anionic amino acid sequence (SSEEDE, SEQ ID NO:38) to the N-terminus of the hydrophobic peptides both allows synthesis of high purity antigen and ensures effective delivery of the otherwise insoluble antigens to dendritic cells of the immune system through strong interactions with the cationic nanoparticles in suspension. The efficacy of these vaccine formulations in generating T cell responses was measured by dosing HLA-A2 expressing transgenic mice (AAD mice) with two 0.1 ml injections of the vaccine delivered subcutaneously on day 0 and day 7. Negative control groups mice were vaccinated with MUC1 peptides formulated in sucrose buffer. Immune responses specific to vaccine formulation were measured in an ELISPOT assay by enumerating MUC1 antigen-specific T cells, in triplicate wells, in the spleens obtained from vaccinated mice. Each bar in the graph represents the mean SFU per 250,000 splenocytes for a group of vaccinated mice. Error bars represent mean±SEM for five mice in each group. For identifying MUC1-specific T cells, splenocytes were stimulated with peptide sequences C1A (SEQ ID NO:9: YLAIVYLIAL), C2A (SEQ ID NO:10), V1A (SEQ ID NO:13), V2A (SEQ ID NO:14) C3A (SEQ ID NO:12) and p93L (SEQ ID NO:11).

### Example 3

Effect of the Formulation within R-DOTAP Cationic Surface Charged Liposomal Nanoparticles

The two highly hydrophobic antigens were inserted into the nonpolar interior membrane bilayer of cationic surface charged liposomal nanoparticles formed from the unique enantiomeric immunostimulatory lipid R-DOTAP. This unique delivery vehicle effectively trapped the water-insoluble peptide antigens and delivered them quantitatively in the highly charged water-compatible immunostimulatory nanoparticles to dendritic cells of the immune system where the antigens are displayed on the surface MHC of the activated dendritic cells to induce high levels of antigen-specific cytotoxic T-cells (FIGS. 1, 2 and 3).

As shown in FIG. 1 HLA-A2 specific CD8 T cell peptide antigens derived from MUC1 proteins incorporated into R-DOTAP nanoparticles, show effective immunoactivity of the insoluble C1A (SEQ ID NO:9) when inserted into the lipid bilayer of the liposomal nanoparticle, thus delivering the low aqueous solubility antigen into immune activating cells with the R-DOTAP immunostimulatory. Control peptide antigen formulations were prepared with 0.5 ml of Montanide plus CpG, or “NCI adjuvant” formulation consisting of 50 μg/ml GM-CSF, 20 μg/ml IL-12, 0.8 mg/ml HBV core (128-140) peptide in incomplete Freund's adjuvant. The efficacy of these vaccine formulations in generating T cell responses was measured by dosing HLA-A2 expressing transgenic mice (AAD mice) with two 0.1 ml injections of the vaccine delivered subcutaneously on day 0 and day 7. Control group mice were vaccinated with MUC1 peptides formulated in sucrose buffer alone. Immune responses specific to vaccine formulation were measured in an ELISPOT assay by enumerating MUC1 antigen-specific T cells, in triplicate wells, in the spleens obtained from vaccinated mice. Each data point in the graph represents the mean SFU per 250,000 splenocytes in vaccinated mice. Error bars represent mean±SEM for five mice in each group. For identifying MUC1-specific T cells, splenocytes were stimulated with peptide sequences C1A (SEQ ID NO:9: YLAIVYLIAL).

As illustrated in FIG. 2, HLA-A2 specific CD8 T cell peptide antigens derived from MUC1 proteins incorporated into R-DOTAP nanoparticles, show effective immunoactivity of the insoluble C2A (SEQ ID NO:10) when inserted into the lipid bilayer of the liposomal nanoparticle, thus delivering the low aqueous solubility antigen into immune activating cells with the R-DOTAP immunostimulator. Control peptide antigen formulations were prepared with 0.5 ml of Montanide plus CpG, or “NCI adjuvant” formulation consisting of 50 μg/ml GM-CSF, 20 μg/ml IL-12, 0.8 mg/ml HBV core (128-140) peptide in incomplete Freund's adjuvant. The efficacy of these vaccine formulations in generating T cell responses was measured by dosing HLA-A2 expressing transgenic mice (AAD mice) with two 0.1 ml injections of the vaccine delivered subcutaneously on day 0 and day 7. Control group mice were vaccinated with MUC1 peptides formulated in sucrose buffer alone. Immune responses specific to vaccine formulation were measured in an ELISPOT assay by enumerating MUC1 antigen-specific T cells, in triplicate wells, in the spleens obtained from vaccinated mice. Each data point in the graph represents the mean SFU per 250,000 splenocytes in vaccinated mice. Error bars represent mean±SEM for five mice in each group. For identifying MUC1-specific T cells, splenocytes were stimulated with peptide sequences C2A (SEQ ID NO:10: YLIALAVCQV).

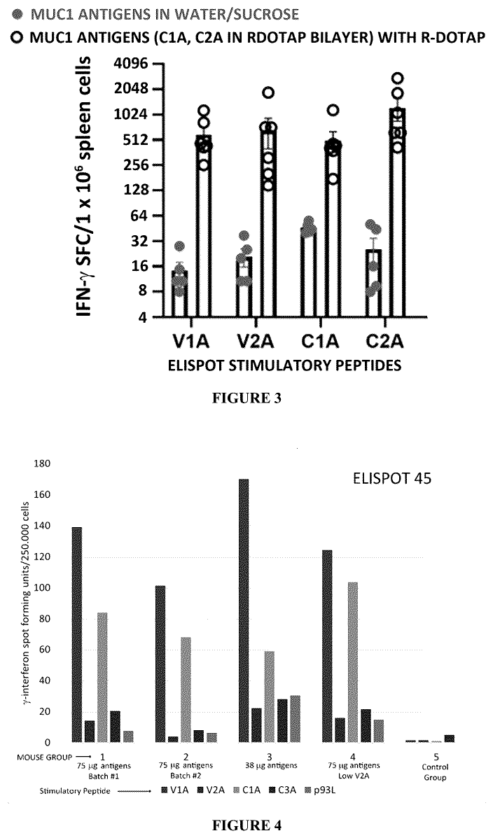

As illustrated in FIG. 3, HLA-A2 specific CD8 T cell responses to formulations including peptide antigen C1A (SEQ ID NO:9) and C2A (SEQ ID NO:10) incorporated into the lipid bilayer of R-DOTAP nanoparticles were assessed. Highly hydrophobic peptide antigens C1A and C2A are ordinarily inactive in generating immune responses when included in antigen mixtures formulated with R-DOTAP immunostimulatory nanoparticles. Insertion of the hydrophobic peptides into the nanoparticle lipid bilayer ensures effective delivery of the otherwise insoluble antigens to dendritic cells of the immune system, generating strong immune responses equivalent to the strong antigens V1A (SEQ ID NO: 13) and V2A (SEQ ID NO:14) in the micellar peptide antigen component of the vaccine. Lipidated peptide agonist antigens VIA and V2A (0.2-1.0 mg/peptide) forming a micellar mixture and agonist antigen C1A and C2A incorporated into the liposome bilayer of R-DOTAP were formulated with 3 mg of R-DOTAP in a 1 ml formulation. The efficacy of these vaccine formulations in generating T cell responses was measured by dosing HLA-A2 expressing transgenic mice (AAD mice) with two 0.1 ml injections of the vaccine delivered subcutaneously on day 0 and day 7. Negative control groups mice were vaccinated with MUC1 peptides formulated in sucrose buffer. Immune responses specific to vaccine formulation were measured in an ELISPOT assay by enumerating MUC1 antigen-specific T cells, in triplicate wells, in the spleens obtained from vaccinated mice. Each data point in the graph represents the mean SFU per million splenocytes in vaccinated mice. Error bars represent mean±SEM for five mice in each group. For identifying MUC1-specific T cells, splenocytes were stimulated with a mixture of peptide sequences C1A (SEQ ID NO:9: YLAIVYLIAL), C2A (SEQ ID NO:10: YLIALAVCQV), V1A (SEQ ID NO:13 YLAPPAHGV), V2A (SEQ ID NO:14: YLDTRPAPV)

This entrapment and delivery of the nonpolar peptide antigens in the enantiomeric cationic lipid nanoparticle lipid bilayer results in very high levels of induction of antigen specific T-cells in murine humanized HLA-A2 transgenic mouse ELISpot assays (FIGS. 1, 2 and 3).

### Example 4

Effect of the Incorporation of the Epitope Peptides into Multiepitope Peptide

The incorporation of the C1A and C2A amino acid sequences into a much larger peptide derived from the sequences of the MUC 1 oncoprotein allowed their effective association and delivery with R-DOTAP immunostimulatory nanoparticles in an aqueous environment. This large peptide, YL40 (SEQ ID NO:1) contains both the hydrophobic agonist peptides on the N-terminus of the molecule where they can be effectively processed by intracellular proteases in dendritic cells to yield the individual agonist peptide antigens for display on cell surface MHC. This approach has been found to yield high level detection of peptide agonist C1A specific T-cell responses after humanized transgenic HLA-A mouse vaccination and ELISpot analysis (FIGS. 6 AND 7).

As shown in FIG. 6, HLA-A2 specific CD8 T cell responses to MUC1/R-DOTAP vaccine formulations containing long MUC1 peptide antigen YL-40, containing the C1A (SEQ ID NO:2) and C2A (SEQ ID NO:3) antigens were assessed. Inclusion of peptide YL40 (SEQ ID NO:1) yielded strong antigen specific responses to antigens C1A and C2A. Lipidated peptide antigen agonists (0.2-1.0 mg/peptide) derived from MUC1 were formulated as peptide micelles with 3 mg of R-DOTAP in a 1 ml formulation along with long peptide antigen YL-40. The efficacy of these vaccine formulations in generating T cell responses was measured by dosing HLA-A2 expressing transgenic mice (AAD mice) with two 0.1 ml injections of the vaccine delivered subcutaneously on day 0 and day 7. Control group mice were vaccinated with MUC1 peptides formulated in sucrose/water solution. Immune responses specific to vaccine formulations were measured in an ELISPOT assay by enumerating MUC1 antigen-specific T cells, in triplicate wells, in the spleens obtained from vaccinated mice. Each data point in the graph represents the mean SFU per million splenocytes in vaccinated mice. Error bars represent mean±SEM for five mice in each group. For identifying MUC1-specific T cells, splenocytes were stimulated with a mixture of peptide sequences C1A (SEQ ID NO:9: YLAIVYLIAL), C2A (SEQ ID NO:10: YLIALAVCQV), V1A (SEQ ID NO:13 YLAPPAHGV), V2A (SEQ ID NO:14: YLDTRPAPV).

As illustrated in FIG. 7, HLA-A2 specific CD8 T cell responses to formulations including peptide antigen C1A (SEQ ID NO:9) and C2A (SEQ ID NO:10) incorporated into the lipid bilayer of R-DOTAP nanoparticles were assessed. Highly hydrophobic peptide antigens C1A and C2A are ordinarily inactive in generating immune responses when included in antigen mixtures formulated with R-DOTAP immunostimulatory nanoparticles. Insertion of the hydrophobic peptides into the nanoparticle lipid bilayer ensures effective delivery of the otherwise insoluble antigens to dendritic cells of the immune system, generating strong immune responses equivalent to the strong antigens pV1A (SEQ ID NO:2) and pV2A (SEQ ID NO:3) in the micellar peptide antigen component of the vaccine. Lipidated peptide agonist antigens (0.2-1.0 mg/peptide) forming a micellar mixture and agonist antigen C1A and C2A incorporated into the liposome bilayer of R-DOTAP were formulated with 3 mg of R-DOTAP in a 1 ml formulation. The efficacy of these vaccine formulations in generating T cell responses was measured by dosing HLA-A2 expressing transgenic mice (AAD mice) with two 0.1 ml injections of the vaccine delivered subcutaneously on day 0 and day 7. Negative control groups mice were vaccinated with MUC1 peptides formulated in sucrose buffer. Immune responses specific to vaccine formulation were measured in an ELISPOT assay by enumerating MUC1 antigen-specific T cells, in triplicate wells, in the spleens obtained from vaccinated mice. Each data point in the graph represents the mean SFU per million splenocytes in vaccinated mice. Error bars represent mean±SEM for five mice in each group. For identifying MUC1-specific T cells, splenocytes were stimulated with a mixture of peptide sequences C1A (SEQ ID NO:9: YLAIVYLIAL), C2A (SEQ ID NO:10: YLIALAVCQV), V1A (SEQ ID NO:13 YLAPPAHGV), V2A (SEQ ID NO:14: YLDTRPAPV).

### Example 5

### Effect of Mixture Versus Encapsulation on the Induction of the Immune Response

Numerous combinations of MUC1 peptide sequences including short specific peptide antigens and other long overlapping peptide sequences from the MUC1 protein have been generated and demonstrated to be ineffective when combined with or incorporated into R-DOTAP immunostimulatory nanoparticles.

For example, and as illustrated in FIG. 8, while the YL40 (SEQ ID NO:1) long peptide induces a significant C1A response (activity), the YL35 (SEQ ID NO:40) peptide did not generate a significant C2A response (using the same approach as YL40); YL35 did induce an immune response to C2A but it was weak. The long peptide sequence YL40 enabled a strong immune response to the C-terminus specifically C1A when mixed with R-DOTAP. The long peptide YL35 which includes the C2A sequence did not enable a strong immune response when mixed with R-DOTAP. This indicates that not all long peptides have the ability to promote a strong immune response against the incorporated sequence even when mixed with R-DOTAP.

Further, it was demonstrated that the short C1A and C2A peptides mixed with R-DOTAP did not generate a significant response, but that the encapsulation of the short C1A and C2A peptides did generate a significant response. The short peptides C1A and C2A only induced an immune response when they were incorporated into the R-DOTAP bilayer, but not when mixed with R-DOTAP.

FIG. 8 also shows that as expected, short peptides without R-DOTAP have no activity

Although the invention has been described with reference to the above examples, it will be understood that modifications and variations are encompassed within the spirit and scope of the invention. Accordingly, the invention is limited only by the following claims.

1. A multiepitope peptide comprising at least 80% sequence identity to the amino acid sequence comprising SEQ ID NO:1.

2. The multiepitope peptide of claim 1, wherein the multiepitope peptide comprises at least 80%, sequence identity to the amino acid sequence comprising any of SEQ ID NOs:9-14 and 20-37.

3. The multiepitope peptide of claim 1, wherein the multiepitope peptide comprises at least one mucin 1 (MUC1) peptide.

4-10. (canceled)

11. A composition comprising a multiepitope peptide and a cationic lipid, wherein the multiepitope peptide comprises at least one mucin 1 (MUC1) peptide.

12. The composition of claim 11, wherein the multiepitope peptide comprises at least 80%, sequence identity to the amino acid sequence of SEQ ID NO:1.

13. (canceled)

14. The composition of claim 11, wherein the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof.

15-20. (canceled)

21. The composition of claim 11, wherein the multiepitope peptide is encapsulated in liposomes comprising cationic lipids or are mixed as micelles with preformed cationic lipid nanoparticles.

22-23. (canceled)

24. The composition of claim 11, further comprising an enhancer agonist epitope and/or an analog thereof.

25. A vaccine composition comprising:
(a) a multiepitope peptide, wherein the multiepitope peptide comprises at least one mucin 1 (MUC1) peptide; and
(b) a cationic lipid.

26. The vaccine composition of claim 25, wherein the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof.

27. (canceled)

28. The vaccine composition of claim 25, wherein the multiepitope peptide comprises a sequence comprising at least 80% sequence identity to the amino acid sequence of SEQ ID NO: 1.

29-34. (canceled)

35. The vaccine composition of claim 25, wherein the multiepitope peptide comprises a sequence of SEQ ID NO:1.

36. The vaccine composition of claim 35, wherein the multiepitope peptide comprises an amino acid sequence comprising at least 80% identity to SEQ ID NOs:18, 19, 42 or 43.

37. The vaccine composition of claim 25, wherein the multiepitope peptide is encapsulated in liposomes comprising cationic lipids or is mixed as separate micelles with preformed cationic lipid nanoparticles.

38-40. (canceled)

41. A method of treating cancer in a subject comprising administering to the subject a vaccine composition of claim 25
thereby treating cancer in the subject.

42-48. (canceled)

49. The method of claim 41, wherein one or more MUC1 peptides of the vaccine composition induce the presentation of non-HLA restricted peptides to CD4⁺ and CD8⁺ T cells by antigen presenting cells.

50. (canceled)

51. The method of claim 41, wherein the cancer comprises MUC1 expressing cancer cells.

52. The method of claim 41, wherein the cancer is prostate cancer, breast cancer or acute myeloid leukemia (AML).

53. The method of claim 41, further comprising administering to the subject an anti-cancer treatment.

54. The method of claim 53, wherein the anti-cancer treatment comprises an immune checkpoint inhibitor therapy.

55. (canceled)

56. A method of inducing a MUC-specific polyfunctional cytolytic T cell response in the subject comprising administering to the subject a composition of claim 11 thereby inducing a MUC-specific polyfunctional cytolytic T cell response in the subject.

57-63. (canceled)

64. The method of claim 56, wherein the multiepitope peptide induces the presentation of non-HLA restricted peptides to CD4⁺ and CD8⁺ T cells by antigen presenting cells.


SEQUENCE LISTING (43 sequences)

SEQ ID NO: 1 - protein, 40 aa, synthetic construct
YLAIVYLIALAVCQVRRKNYGQLDIFPARDKYHPMSEYAL

SEQ ID NO: 2 - protein, 9 aa, synthetic construct
  SITE 1: note = Epsilon-palmitoyl modified residue
YLAPPAHGV

SEQ ID NO: 3 - protein, 9 aa, synthetic construct
  SITE 1: note = Epsilon-palmitoyl modified residue
YLDTRPAPV

SEQ ID NO: 4 - protein, 9 aa, synthetic construct
  SITE 1: note = Epsilon-palmitoyl modified residue
SLFRSPYEK

SEQ ID NO: 5 - protein, 10 aa, synthetic construct
  SITE 1: note = Epsilon-palmitoyl modified residue
KYHPMSEYAL

SEQ ID NO: 6 - protein, 9 aa, synthetic construct
  SITE 1: note = Epsilon-palmitoyl modified residue
KYTNPAVAL

SEQ ID NO: 7-8 - skipped (2 entries)

SEQ ID NO: 9 - protein, 10 aa, synthetic construct
YLAIVYLIAL

SEQ ID NO: 10 - protein, 10 aa, synthetic construct
YLIALAVCQV

SEQ ID NO: 11 - protein, 10 aa, synthetic construct
ALWGQDVTSV

SEQ ID NO: 12 - protein, 9 aa, synthetic construct
YLSYTNPAV

SEQ ID NO: 13 - protein, 9 aa, synthetic construct
YLAPPAHGV

SEQ ID NO: 14 - protein, 9 aa, synthetic construct
YLDTRPAPV

SEQ ID NO: 15 - protein, 9 aa, synthetic construct
  SITE 1: note = Epsilon-palmitoyl modified residue
YLSYTNPAV

SEQ ID NO: 16 - protein, 10 aa, synthetic construct
  SITE 1: note = Epsilon-palmitoyl modified residue
ALWGQDVTSV

SEQ ID NO: 17 - protein, 10 aa, synthetic construct
  SITE 1: note = Epsilon-palmitoyl modified residue
ALFIVYLIAK

SEQ ID NO: 18 - protein, 16 aa, synthetic construct
SSEEDEYLAIVYLIAL

SEQ ID NO: 19 - protein, 16 aa, synthetic construct
SSEEDEYLIALAVCQV

SEQ ID NO: 20 - protein, 10 aa, synthetic construct
YLAIVTLIAL

SEQ ID NO: 21 - protein, 10 aa, synthetic construct
YLIALAVCQV

SEQ ID NO: 22 - protein, 9 aa, synthetic construct
YLAPPAHGV

SEQ ID NO: 23 - protein, 9 aa, synthetic construct
YLDTRPAPV

SEQ ID NO: 24 - protein, 9 aa, synthetic construct
YLSYTNPAV

SEQ ID NO: 25 - protein, 10 aa, synthetic construct
ALFIVYLIAK

SEQ ID NO: 26 - protein, 9 aa, synthetic construct
SLFRSPYEK

SEQ ID NO: 27 - protein, 10 aa, synthetic construct
KYHPMSEYAL

SEQ ID NO: 28 - protein, 9 aa, synthetic construct
KYTNPAVAL

SEQ ID NO: 29 - protein, 10 aa, synthetic construct
ALAIVYLIAL

SEQ ID NO: 30 - protein, 10 aa, synthetic construct
YLIALAVCQC

SEQ ID NO: 31 - protein, 9 aa, synthetic construct
STAPPAHGV

SEQ ID NO: 32 - protein, 9 aa, synthetic construct
APDTRPAPG

SEQ ID NO: 33 - protein, 9 aa, synthetic construct
SLSYTNPAV

SEQ ID NO: 34 - protein, 10 aa, synthetic construct
ALAIVYLIAL

SEQ ID NO: 35 - protein, 9 aa, synthetic construct
STDRSPYEK

SEQ ID NO: 36 - protein, 10 aa, synthetic construct
TYHPMSEYPT

SEQ ID NO: 37 - protein, 9 aa, synthetic construct
SYTNPAVAA

SEQ ID NO: 38 - protein, 6 aa, synthetic construct
SSEEDE

SEQ ID NO: 39 - protein, 7 aa, synthetic construct
SSEEDEE

SEQ ID NO: 40 - protein, 35 aa, synthetic construct
YLIALAVCQVRRKNYGQLDIFPARDKYHPMSEYAL

SEQ ID NO: 41 - protein, 33 aa, synthetic construct
STDRSPYEKVSAGNGGSSLSYTNPAVAATSANL

SEQ ID NO: 42 - protein, 17 aa, synthetic construct
SSEEDEEYLAIVYLIAL

SEQ ID NO: 43 - protein, 17 aa, synthetic construct
SSEEDEEYLIALAVCQV